(19) United States
(12) Patent Application Publication
(10) Pub. No.: US 2003/0228379 A1
(43) Pub. Date: Dec. 11, 2003

(54) Herbs and herbal combinations useful for the treatment of microbial infections

(76) Inventors: Wenyuan Shi, Los Angeles, CA (US); Li Chen, Los Angeles, CA (US); No-Hee Park, Los Angeles, CA (US); Maxwell H. Anderson, Seattle, WA (US)

Correspondence Address: GRAY CARY WARE & FREIDENRICH LLP, 153 TOWNSEND, SUITE 800, SAN FRANCISCO, CA, 94107, US

(21) Appl. No.: 10/156,278

(22) Filed: May 28, 2002

(51) Int. Cl. A61K 35/78

(52) U.S. Cl. 424/725

(57) ABSTRACT

The present invention provides compositions of herbs and methods of using the compositions for treating and preventing microbial infection, especially dental caries or periodontal disease.

### FIELD OF THE INVENTION

This invention relates generally to the field of herbs, and more specifically to Chinese herbs useful for the treatment of microbial infections.

### BACKGROUND OF THE INVENTION

Modern medical science is constantly searching for new and more powerful agents to prevent, treat or retard bacterial and viral infections and cure the diseases they cause. Bacterial and viral infections of humans and domestic animals cost billions of dollars annually. Vast sums of money are spent each year by pharmaceutical companies to identify, characterize, and produce new antibiotics and antivirals to combat the emerging drug resistant strains which have become a serious problem. Reliable prophylactic treatments for disease prevention are also of major interest.

Specifically periodontal disease and dental caries are of major public health and economic interest worldwide. It is now widely recognized that both of these oral diseases are caused by bacteria which grow in masses on the teeth and in the gingival and subgingival areas. A commonly used descriptive term for these bacterial masses is “dental plaque”. In the case of periodontal disease, it has been reported that dental plaque bacteria, growing in the area where the teeth and gingival tissues meet, cause an inflammation of the gingiva called “gingivitis”. This is characterized by swollen, edematous gingiva (“gums”) which are reddened and bleed easily. If plaque removal is inadequate, gingivitis may progress to “periodontitis” or periodontal disease in some individuals. Periodontitis generally is characterized by a chronic inflammation of the tissues around the teeth, which leads to a resorption of supporting bone. Periodontal disease is the leading cause of tooth loss among adults.

Dental caries (cavities) are also caused by bacteria, with mutans Streptococcus being the principal etiologic agent. Dental caries is a prevalent and costly disease throughout the world. The latest report by NIH indicated that 49% of 12-year-old and 79% of 17-year-old children in the USA have dental caries. A very high percentage of the elderly also have tooth decay manifest as root caries.

Tooth decay is mainly caused by a group of cariogenic Gram-positive bacteria such as Streptococcus mutans. Given a suitable carbohydrate nutrient (simple dimer sugars like sucrose), these bacteria produce insoluble glucans and acids in dental plaque. The glucans produced by S. mutans are very sticky, enabling it to adhere to the tooth's surface while the acids attack the tooth's mineral structure causing demineralization that may lead to cavitation.

The prevention of dental plaque or the removal thereof has long been the focus of development, with the ultimate goal of inhibiting both caries and periodontal diseases. While the formation of dental plaque can be inhibited to a certain extent by brushing the teeth at frequent intervals, brushing alone is not sufficient to effectively prevent the formation of dental plaque or remove substantially all of the dental plaque that has formed on the teeth. Since brushing alone is often not sufficient to prevent dental caries or periodontal disease due to the nature of the pathogenic plaque bacteria, chemical methods using anti-bacterials such as chlorhexidine, benzalkonium chloride, and cetylpyridinium chloride have been proposed.

There is a need in the art to provide compositions or products useful for treating or preventing microbial conditions, e.g., oral microbial conditions such as periodontal disease and dental caries.

### SUMMARY OF THE INVENTION

The present invention is based on the discovery that a pool of natural herbs or the combinations thereof have anti-microbial activity, e.g., anti-bacterial, anti-fungus activity, or ability of disrupting bacterial quorum sensing. Accordingly, the present invention provides compositions of herbal combinations useful for treating or preventing microbial conditions, e.g., oral microbial conditions such as periodontal disease and dental caries. The present invention also provides methods of using herbs and the combinations thereof to treat or prevent microbial conditions, e.g., oral microbial conditions such as periodontal disease and dental caries. In addition, the present invention provides herbal libraries useful for screening or identifying combinations of herbs with desired activities, e.g., against many microbial forms.

In one embodiment, the present invention provides a composition comprising a mixture of at least two components selected from the group consisting of phellodendron amurense, Paris polyphylla Smith, Prunus mume (sieb.), glycyrrhiza uralensis Fisch, Amomum villosum, Sanguisorba officinalis, Elsholtzia splendens, Eugenia caryophyllata, Rhus chinensis mill, Atractylodes chinensis koidz, perilla frutescens (Britt), Coptis chinensis franch, Sophora flavescens Ait, Bletilla striata (thunb), Amomum cardamomum (karvanh), Sophora tonkinensis (subprostrata), Melia toosendan, and Medicinal rhubarb root.

In another embodiment, the present invention provides a composition comprising a mixture of at least three components selected from the group consisting of phellodendron amurense, Paris polyphylla Smith, Prunus mume (sieb.), glycyrrhiza uralensis Fisch, Amomum villosum, Sanguisorba officinalis, Elsholtzia splendens, Eugenia caryophyllata, Rhus chinensis mill, Atractylodes chinensis koidz, perilla frutescens (Britt), Coptis chinensis franch, Sophora flavescens Ait, Bletilla striata (thunb), Amomum cardamomum (karvanh), Sophora tonkinensis (subprostrata), Melia toosendan, and Medicinal rhubarb root.

In yet another embodiment, the present invention provides a composition comprising a mixture of at least four components selected from the group consisting of phellodendron amurense, Paris polyphylla Smith, Prunus mume (sieb.), glycyrrhiza uralensis Fisch, Amomum villosum, Sanguisorba officinalis, Elsholtzia splendens, Eugenia caryophyllata, Rhus chinensis mill, Atractylodes chinensis koidz, perilla frutescens (Britt), Coptis chinensis franch, Sophora flavescens Ait, Bletilla striata (thunb), Amomum cardamomum (karvanh), Sophora tonkinensis (subprostrata), Melia toosendan, and Medicinal rhubarb root.

In still another embodiment, the present invention provides a composition comprising a mixture of component A selected from the group consisting of Paris polyphylla Smith, Prunus mume (sieb.), glycyrrhiza uralensis Fisch, Amomum villosum, Sanguisorba officinalis, Elsholtzia splendens, Eugenia caryophyllata, Rhus chinensis mill, Atractylodes chinensis koidz, perilla frutescens (Britt), Coptis chinensis franch, Sophora flavescens Ait, Bletilla striata (thunb), Amomum cardamomum (karvanh), Sophora tonkinensis (subprostrata), and Melia toosendan, and component B selected from the group consisting of Prunus mume (sieb.), glycyrrhiza uralensis Fisch, Amomum villosum, Sanguisorba officinalis, Elsholtzia splendens, Rhus chinensis mill, Atractylodes chinensis koidz, perilla frutescens (Britt), Coptis chinensis franch, and Sophora flavescens Ait.

In another embodiment, the present invention provides a composition comprising a mixture of component A selected from the group consisting of Paris polyphylla Smith, Prunus mume (sieb.), glycyrrhiza uralensis Fisch, Amomum villosum, Sanguisorba officinalis, Elsholtzia splendens, Eugenia caryophyllata, Rhus chinensis mill, Atractylodes chinensis koidz, perilla frutescens (Britt), Coptis chinensis franch, Sophora flavescens Ait, Bletilla striata (thunb), Amomum cardamomum (karvanh), Sophora tonkinensis (subprostrata), and Melia toosendan, and component B selected from the group consisting of phellodendron amurense, Paris polyphylla Smith, and Sophora flavescens Ait.

In another embodiment, the present invention provides a composition comprising a mixture of component A selected from the group consisting of Paris polyphylla Smith, Prunus mume (sieb.), glycyrrhiza uralensis Fisch, Amomum villosum, Sanguisorba officinalis, Elsholtzia splendens, Eugenia caryophyllata, Rhus chinensis mill, Atractylodes chinensis koidz, perilla frutescens (Britt), Coptis chinensis franch, Sophora flavescens Ait, Bletilla striata (thunb), Amomum cardamomum (karvanh), Sophora tonkinensis (subprostrata), and Melia toosendan, and component B selected from the group consisting of Coptis chinensis franch, Sophora flavescens Ait, and Medicinal rhubarb root.

In another embodiment, the present invention provides a composition comprising a mixture of component A selected from the group consisting of Prunus mume (sieb.), glycyrrhiza uralensis Fisch, Amomum villosum, Sanguisorba officinalis, Elsholtzia splendens, Rhus chinensis mill, Atractylodes chinensis koidz, perilla frutescens (Britt), Coptis chinensis franch, and Sophora flavescens Ait, and component B selected from the group consisting of phellodendron amurense, Paris polyphylla Smith, and Sophora flavescens Ait.

In another embodiment, the present invention provides a composition comprising a mixture of component A selected from the group consisting of Prunus mume (sieb.), glycyrrhiza uralensis Fisch, Amomum villosum, Sanguisorba officinalis, Elsholtzia splendens, Rhus chinensis mill, Atractylodes chinensis koidz, perilla frutescens (Britt), Coptis chinensis franch, and Sophora flavescens Ait, and component B selected from the group consisting of Coptis chinensis franch, Sophora flavescens Ait, and Medicinal rhubarb root.

In another embodiment, the present invention provides a composition comprising a mixture of component A selected from the group consisting of phellodendron amurense, Paris polyphylla Smith, and Sophora flavescens Ait, and component B selected from the group consisting of Coptis chinensis franch, Sophora flavescens Ait, and Medicinal rhubarb root.

In another embodiment, the present invention provides a composition comprising a mixture of component A selected from the group consisting of Paris polyphylla Smith, Prunus mume (sieb.), glycyrrhiza uralensis Fisch, Amomum villosum, Sanguisorba officinalis, Elsholtzia splendens, Eugenia caryophyllata, Rhus chinensis mill, Atractylodes chinensis koidz, perilla frutescens (Britt), Coptis chinensis franch, Sophora flavescens Ait, Bletilla striata (thunb), Amomum cardamomum (karvanh), Sophora tonkinensis (subprostrata), and Melia toosendan, component B selected from the group consisting of Prunus mume (sieb.), glycyrrhiza uralensis Fisch, Amomum villosum, Sanguisorba officinalis, Elsholtzia splendens, Rhus chinensis mill, Atractylodes chinensis koidz, perilla frutescens (Britt), Coptis chinensis franch, and Sophora flavescens Ait, and component C selected from the group consisting of phellodendron amurense, Paris polyphylla Smith, and Sophora flavescens Ait.

In another embodiment, the present invention provides a composition comprising a mixture of component A selected from the group consisting of Prunus mume (sieb.), glycyrrhiza uralensis Fisch, Amomum villosum, Sanguisorba officinalis, Elsholtzia splendens, Rhus chinensis mill, Atractylodes chinensis koidz, perilla frutescens (Britt), Coptis chinensis franch, and Sophora flavescens Ait, component B selected from the group consisting of phellodendron amurense, Paris polyphylla Smith, and Sophora flavescens Ait, and component C selected from the group consisting of Coptis chinensis franch, Sophora flavescens Ait, and Medicinal rhubarb root.

In another embodiment, the present invention provides a composition comprising a mixture of Sophora flavescens Ait, Paris polyphylla Smith, perilla frutescens (Britt), and Coptis chinensis franch.

In yet another embodiment, the present invention provides a composition comprising a mixture of glycyrrhiza uralensis Fisch, Paris polyphylla Smith, perilla frutescens (Britt), and Coptis chinensis franch.

In still another embodiment, the present invention provides a composition comprising a mixture of Elsholtzia splendens, Paris polyphylla Smith, perilla frutescens (Britt), and Coptis chinensis franch.

In another embodiment, the present invention provides a method of inhibiting the activity of a microorganism. The method comprises contacting the microorganism to a composition comprising a component selected from the group consisting of phellodendron amurense, Paris polyphylla Smith, Prunus mume (sieb.), glycyrrhiza uralensis Fisch, Amomum villosum, Sanguisorba officinalis, Elsholtzia splendens, Eugenia caryophyllata, Rhus chinensis mill, Atractylodes chinensis koidz, perilla frutescens (Britt), Coptis chinensis franch, Sophora flavescens Ait, Bletilla striata (thunb), Amomum cardamomum (karvanh), Sophora tonkinensis (subprostrata), Melia toosendan, and Medicinal rhubarb root.

In yet another embodiment, the present invention provides a method of treating a microbial infection comprising administering to a subject in need of such treatment a composition comprising a component selected from the group consisting of phellodendron amurense, Paris polyphylla Smith, Prunus mume (sieb.), glycyrrhiza uralensis Fisch, Amomum villosum, Sanguisorba officinalis, Elsholtzia splendens, Eugenia caryophyllata, Rhus chinensis mill, Atractylodes chinensis koidz, perilla frutescens (Britt), Coptis chinensis franch, Sophora flavescens Ait, Bletilla striata (thunb), Amomum cardamomum (karvanh), Sophora tonkinensis (subprostrata), Melia toosendan, and Medicinal rhubarb root.

In another embodiment, the present invention provides a method of preventing a microbial infection. The method comprises contacting a composition to an area susceptible to a microorganism causing the microbial infection, wherein the composition comprises a component selected from the group consisting of phellodendron amurense, Paris polyphylla Smith, Prunus mume (sieb.), glycyrrhiza uralensis Fisch, Amomum villosum, Sanguisorba officinalis, Elsholtzia splendens, Eugenia caryophyllata, Rhus chinensis mill, Atractylodes chinensis koidz, perilla frutescens (Britt), Coptis chinensis franch, Sophora flavescens Ait, Bletilla striata (thunb), Amomum cardamomum (karvanh), Sophora tonkinensis (subprostrata), Melia toosendan, and Medicinal rhubarb root.

In yet another embodiment, the present invention provides a method of preventing a microbial infection. The method comprises administering to a subject in need of such treatment a composition comprising a component selected from the group consisting of phellodendron amurense, Paris polyphylla Smith, Prunus mume (sieb.), glycyrrhiza uralensis Fisch, Amomum villosum, Sanguisorba officinalis, Elsholtzia splendens, Eugenia caryophyllata, Rhus chinensis mill, Atractylodes chinensis koidz, perilla frutescens (Britt), Coptis chinensis franch, Sophora flavescens Ait, Bletilla striata (thunb), Amomum cardamomum (karvanh), Sophora tonkinensis (subprostrata), Melia toosendan, and Medicinal rhubarb root.

In still another embodiment, the present invention provides an herbal library consisting essentially of phellodendron amurense, Paris polyphylla Smith, Prunus mume (sieb.), glycyrrhiza uralensis Fisch, Amomum villosum, Sanguisorba officinalis, Elsholtzia splendens, Eugenia caryophyllata, Rhus chinensis mill, Atractylodes chinensis koidz, perilla frutescens (Britt), Coptis chinensis franch, Sophora flavescens Ait, Bletilla striata (thunb), Amomum cardamomum (karvanh), Sophora tonkinensis (subprostrata), Melia toosendan, and Medicinal rhubarb root.

In another embodiment, the present invention provides a composition comprising a mixture of component A selected from the group consisting of Paris polyphylla Smith, Prunus mume (sieb.), glycyrrhiza uralensis Fisch, Amomum villosum, Sanguisorba officinalis, Elsholtzia splendens, Eugenia caryophyllata, Rhus chinensis mill, Atractylodes chinensis koidz, perilla frutescens (Britt), Coptis chinensis franch, Sophora flavescens Ait, Bletilla striata (thunb), Amomum cardamomum (karvanh), Sophora tonkinensis (subprostrata), and Melia toosendan, component B selected from the group consisting of Prunus mume (sieb.), glycyrrhiza uralensis Fisch, Amomum villosum, Sanguisorba officinalis, Elsholtzia splendens, Rhus chinensis mill, Atractylodes chinensis koidz, perilla frutescens (Britt), Coptis chinensis franch, and Sophora flavescens Ait, and component C selected from the group consisting of Coptis chinensis franch, Sophora flavescens Ait, and Medicinal rhubarb root.

In yet another embodiment, the present invention provides a composition comprising a mixture of component A selected from the group consisting of Paris polyphylla Smith, Prunus mume (sieb.), glycyrrhiza uralensis Fisch, Amomum villosum, Sanguisorba officinalis, Elsholtzia splendens, Eugenia caryophyllata, Rhus chinensis mill, Atractylodes chinensis koidz, perilla frutescens (Britt), Coptis chinensis franch, Sophora flavescens Ait, Bletilla striata (thunb), Amomum cardamomum (karvanh), Sophora tonkinensis (subprostrata), and Melia toosendan, component B selected from the group consisting of phellodendron amurense, Paris polyphylla Smith, and Sophora flavescens Ait, and component C selected from the group consisting of Coptis chinensis franch, Sophora flavescens Ait, and Medicinal rhubarb root.

### DESCRIPTION OF THE PREFERRED EMBODIMENTS

The present invention relates in general to herbs and combinations thereof useful for treating or preventing microbial conditions. It is the discovery of the present invention that certain herbs and combinations thereof have anti-microbial activity, e.g., anti-bacterial, anti-fungal activity, or ability of interrupting bacterial quorum sensing. Accordingly, the present invention provides compositions and methods of using the compositions for treating or preventing microbial conditions, e.g., oral microbial conditions such as periodontal disease and dental caries. The present invention also provides herbal libraries useful for identifying combinations of herbs with desired activities or profiling the herbs therein, e.g., profiling antimicrobioal or biochemical activities of the herbs in the libraries.

According to one feature of the present invention, it provides an herbal library, General Herbal Library (GHL), containing phellodendron amurense, Paris polyphylla Smith, Prunus mume (sieb.), glycyrrhiza uralensis Fisch, Amomum villosum, Sanguisorba officinalis, Elsholtzia splendens, Eugenia caryophyllata, Rhus chinensis mill, Atractylodes chinensis koidz, perilla frutescens (Britt), Coptis chinensis franch, Sophora flavescens Ait, Bletilla striata (thunb), Amomum cardamomum (karvanh), Sophora tonkinensis (subprostrata), Melia toosendan and Medicinal rhubarb root. Table 1 describes the Chinese name and common name for each Latin name listed above.

TABLE 1
General Herbal Library

| Chinese Name | Common Name | Latin Name |
|---|---|---|
| Huang-Bai | Amu corktree bark | phellodendron amurense |
| Qi-ye-yi-zhi-hua | manyleaf paris | Paris polyphylla Smith |
| | rhizome | |
| Wu-mei | Japanese apricot fruit | Prunus mume (sieb.) |
| Gan-chao | ural licorice root | glycyrrhiza uralensis Fisch |
| Sai-ren | villous amomum fruit | Amomum villosum |
| Di-yu | garden burnet root | Sanguisorba officinalis |
| Xiang Ru | Elsholtzia | Elsholtzia splendens |
| Ding xiang | clove twig | Eugenia caryophyllata |
| Wu-bei-zi | chinese gall | Rhus chinensis mill |
| Chang-su | swordlike atractylodes | Atractylodes chinensis koidz |
| | rhizome | |
| Zi-su | common perilla leaf | perilla frutescens (Britt) |
| Huang-lian | Chinese goldthread | Coptis chinensis franch |
| | rhizome | |
| Ku-shen | lightyellow sophora | Sophora flavescens Ait |
| | root | |
| Bai-ji | common bletilla tuber | Bletilla striata (thunb) |
| Bai-dou-kou | white amomum fruit | Amomum cardamomum |
| | | (karvanh) |
| Sam-dou-gang | toniken sophora | Sophora tonkinensis |
| | | (subprostrata) |
| chwan lia zi | chinaberry fruit | Melia toosendan |
| Da-huang | Rheum officinale Baill | Medicinal rhubarb root |

In one embodiment, the present invention provides an herbal library containing one or more subgroups of herbs in the GHL. For example, Subgroup One Library (SOL) includes Paris polyphylla Smith, Prunus mume (sieb.), glycyrrhiza uralensis Fisch, Amomum villosum, Sanguisorba officinalis, Elsholtzia splendens, Eugenia caryophyllata, Rhus chinensis mill, Atractylodes chinensis koidz, perilla frutescens (Britt), Coptis chinensis franch, Sophora flavescens Ait, Bletilla striata (thunb), Amomum cardamomum (karvanh), Sophora tonkinensis (subprostrata), and Melia toosendan; Subgroup Two Library (STL) includes Prunus mume (sieb.), glycyrrhiza uralensis Fisch, Amomum villosum, Sanguisorba officinalis, Elsholtzia splendens, Rhus chinensis mill, Atractylodes chinensis koidz, perilla frutescens (Britt), Coptis chinensis franch, and Sophora flavescens Ait; Subgroup Three Library (SThL) includes phellodendron amurense, Paris polyphylla Smith, and Sophora flavescens Ait; while Subgroup Four Library (SFL) includes Coptis chinensis franch, Sophora flavescens Ait, and Medicinal rhubarb root.

In another embodiment, the present invention provides an herbal library with an instruction. For example, the instruction can include an activity profile for each herb of the herbal library or an activity profile for each subgroup, e.g., SOL has anti-G⁺ bacterial activity, STL has anti-G⁻ bacterial activity, SThL has anti-fungus activity, while SFL affect or disrupting bacterial quorum sensing.

The herbal libraries provided by the present invention can be used for various purposes. For example, the herbal libraries can be used as a source of agents having anti-microbial activity or to be screened for additional desired activities, or used for identifying combinations of herbs with desired activities. In particular, activity profiles for each herb of the herbal libraries of the present invention or activity profiles for each subgroup of the GHL, e.g., SOL. STL, SThL or SFL can provide guidance for herbal library screening and identifying useful combinations of herbs.

According to another feature of the present invention, it provides compositions containing as active ingredients a mixture of herbs, e.g., combinations of herbs having anti-microbial activity including without limitation anti-G⁺, anti-G⁻, anti-fungus, or affecting bacterial quorum sensing. For example, the composition of the present invention can contain as active ingredients at least two herbs selected from the General Herbal Library (GHL) of the present invention. In one embodiment, the composition of the present invention contains as active ingredients at least three herbs selected from the GHL. In another embodiment, the composition of the present invention includes as active ingredients at least four herbs selected from the GHL.

Usually the herbs in the composition of the present invention can be selected either generally from the GHL or specifically from any one of the subgroups of GHL, e.g., SOL, STL, SThL, or SFL. In one embodiment, the composition of the present invention contains at least two herbs, with the first herb selected from SOL, STL, SThL, or SFL, and the second herb selected from a subgroup of GHL that is different from the subgroup for the first herb. In another embodiment, the composition of the present invention contains at least three herbs, with the first, second, and the third herb selected from SOL, STL, and SThL, STL, SThL, and SFL, SOL, STL, and SFL, and SOL, SThL, and SFL, respectively. In yet another embodiment, the composition of the present invention contains at least four herbs, with the first, second, third, and forth herb selected from SOL, STL, SThL, and SFL, respectively.

The herbs in the composition of the present invention can have any weight ratios suitable for providing the composition with an anti-microbial activity. One skilled in the art can readily determine such suitable weight ratios by testing anti-microbial activity of compositions of different weight ratios in routine bioassays. Generally the weight ratio for each herb of the composition is from about 1 to about 5, e.g., (1-5):(1-5), (1-5):(1-5):(1-5), and (1-5):(1-5):(1-5):(1-5). In one embodiment, about same amount each herb in the composition of the present invention, e.g., about equal ratio for each herb such as 1:1, 1:1:1, or 1:1:1:1.

In a preferred embodiment, the present invention provides a composition comprising as active ingredients a mixture of Sophora flavescens Ait, Paris polyphylla Smith, perilla frutescens (Britt), and Coptis chinensis franch, e.g. with a weight ratio of about 5:2:2:1. In another preferred embodiment, the present invention provides a composition comprising as active ingredients a mixture of glycyrrhiza uralensis Fisch, Paris polyphylla Smith, perilla frutescens (Britt), and Coptis chinensis franch or Elsholtzia splendens, Paris polyphylla Smith, perilla frutescens (Britt), and Coptis chinensis franch, both of which with a suitable weight ratio of, e.g., about 5:2:2:1.

The herbs of the present invention can be in any form suitable for a desired usage. For example, the herbs of the present invention can be naturally existing herbs, dehydrated herbs, extraction elute of the herbs including dried or liquid extraction elutes, or active ingredient(s) or components of the herbs.

According to another feature of the present invention, it provides a comprehensive anti-microbial composition comprising an anti-G⁺ bacterial agent, an anti-G⁻ bacterial agent, an anti-fungus agent, and an agent capable of interrupting bacterial quorum sensing. Any known or later discovered anti-G⁺ bacterial agent, anti-G⁻ bacterial agent, anti-fungus agent, and agent capable of interrupting bacterial quorum sensing can be used for the comprehensive anti-microbial composition of the present invention. The agents used for the comprehensive anti-microbial composition of the present invention can be any entity having the desired activity. For example, the agents used for the comprehensive anti-microbial composition of the present invention can be chemical compounds, polypeptides, polynucleotides, small molecules, recombinant materials, herbs, natural substance, or any synthetic substances.

The composition of the present invention can also include one or more other non-active ingredients, e.g., ingredients that do not interfere with the function of the active ingredients. For example, the composition of the present invention can include a suitable carrier or be combined with other therapeutic agents.

A suitable carrier can be an aqueous carrier including any safe and effective materials for use in the compositions of the present invention. In one embodiment, an aqueous carrier is used for the compositions of the present invention in oral formations and includes, without limitation, thickening materials, humectants, water, buffering agents, abrasive polishing materials, surfactants, titanium dioxide, flavor system, sweetening agents, coloring agents, and mixtures thereof.

A suitable carrier can also be a pharmaceutically acceptable carrier that is well known to those in the art. Such carriers include, without limitation, large, slowly metabolized macromolecules, e.g., proteins, polysaccharides, polylactic acids, polyglycolic acids, polymeric amino acids, amino acid copolymers, and inactive virus particles.

Pharmaceutically acceptable salts can also be used in the composition, for example, mineral salts such as sodium or stannous fluorides, or sulfates, as well as the salts of organic acids such as acetates, proprionates, carbonates, malonates, or benzoates. The composition can also contain liquids, e.g., water, saline, glycerol, and ethanol, as well as substances, e.g., wetting agents, emulsifying agents, or pH buffering agents.

The compositions of the present invention usually have an anti-microbial effect, e.g., anti-G⁺ bacteria activity, anti-G⁻ bacteria activity, anti-fungus activity, or effect on bacterial quorum sensing. Methods or assays for testing the anti-microbial activity of a composition are readily available to one skilled in the art. For example, compositions of the present invention can be incubated with a bacterial or fungous culture, and the bacterial or fungous growth can be subsequently examined with a plate reader. Compositions of the present invention can also be examined for their effect on bacterial quorum sensing using either an acyl-homoserine lactone quorum sensing reporter system or a luxS quorum sensing reporter system.

According to another feature of the present invention, the compositions of the present invention can be used to treat or prevent microbial growth or infection, e.g., inhibit the activity of bacteria or fungi in vivo or in vitro. For example, the compositions of the present invention can be used to inhibit microbial flora, especially microbial flora associated with dental structures, e.g., tooth surface or subsurface or caries, e.g., microbial flora associated with demineralized areas, white spots, pits, and fissures. In one embodiment, the compositions of the present invention can be used to inhibit microorganisms including without limitation S. mutans, S. sobrinus, L. acidophilus, L. casei, L. plantarum, A. naeslundii, A. viscosus, Actinobacillus actinomycetemcomitants, Porphyromonas gingivalis, Fusobacterium nucleatum, Treponema denticola, Bacteroides forsythus, Candidas albicans, C. glabrata, C. guilliemondii, C. kefyr, C. krusei, C. stellatoidea and C. tropicalis.

In another embodiment, the composition of the present invention can be used to inhibit the activity of cariogenic bacteria, including without limitation, Mutans streptococci, lactobacilli and actinomyces, e.g., S. mutans, S. sobrinus, A. viscosus, A. naeslundii, L. acidophilus, L. casei, and L. plantarum. In yet another embodiment, the composition of the present invention can be used to inhibit the activity of fungi, e.g., Candidas albicans, C. glabrata, C. guilliemondii, C. kefyr, C. krusei, C. stellatoidea and C. tropicalis.

According to another feature of the present invention, it provides a method of inhibiting the activity of microorganisms from one or more species or preventing a microbial infection by contacting one or more compositions of the present invention to the microorganisms. The present invention also provides a method for treating or preventing a microbial infection by administering to a subject in need of such treatment an effective amount of one or more compositions of the present invention. The subject in need of such treatment can be any suitable subject, e.g., a human or an animal including a domestic animal such as a horse, dog, or cat. The microbial infection can be any infection caused by one or more microorganisms of one or more species including without limitation microbial infections associated with multi-species biofilms.

In generally, an effective amount of the compositions to be administered can be determined on a case-by-case basis. Factors to be considered usually include age, body weight, stage of the condition, other disease conditions, duration of the treatment, and the response to the initial treatment.

Typically, the compositions are prepared as a topical or an injectable, either as a liquid solution or suspension. However, solid forms suitable for solution in, or suspension in, liquid vehicles prior to injection can also be prepared. The composition can also be formulated into an enteric-coated tablet or gel capsule according to known methods in the art.

The compositions of the present invention may be administered in any way which is medically acceptable which may depend on the condition or injury being treated. Possible administration routes include injections, by parenteral routes such as intravascular, intravenous, intraepidural or others, as well as oral, nasal, ophthalmic, rectal, vaginal, topical, or pulmonary, e.g., by inhalation. The compositions may also be directly applied to tissue surfaces. Sustained release, pH dependent release, or other specific chemical or environmental condition mediated release administration is also specifically included in the invention, by such means as depot injections or erodible implants.

In one embodiment, the composition of the present invention can be used to treat or prevent microbial infections associated with epithelial tissues or skins, e.g., wounds, bums, acne, fungus infection on skins such as foot, and other skin conditions or with opportunistic organisms, e.g., opportunistic organisms superinfect a site.

In another embodiment, the composition of the present invention can be used to treat or prevent microbial infections on mucosal surfaces, e.g., mouth, vagina, gastrointestinal (GI) tract, esophageal tract, and respiratory tract. For example, the composition of the present invention can be used to treat or prevent Streptococcus pneumoniae, nontypeable Haemophilius influenza, or Moraxella cararrhalis infection commonly found in acute otitis media (AOM) and otitis media effusion (OME) as complications of upper respiratory infections in young children.

In another example, the composition of the present invention can be used to treat or prevent GI tract infections including without limitation duodenal or gastric ulcers associated with Helicobacter pylori (H. pylori) bacteria infection, campylobacter bacterial infection, diarrhea primarily associated with Campylobacter jejuni, cholera caused by Vibrio cholerae serogroups, salmonellosis caused by bacteria salmonella such as S. Typhimurium and S. Enteritidis, shigellosis caused by bacteria Shigella, e.g., Shigella dysenteriae and traveler's diarrhea caused by enterotoxigenic Escherichia coli (ETEC) and Clostridium difficile infection.

In yet another example, the composition of the present invention can be used to treat yeast or Candida infections (Candidiasis) typically occur either orally (Oropharyngeal Candida or OPC) or vaginally (Vulvovaginal Candida or VVC).

According to another embodiment of the present invention, the compositions of the present invention are used to treat or prevent cariogenic organism infections, e.g., S. mutans infection associated with dental caries, including without limitation tooth surface or subsurface associated with demineralized areas, white spots, pits, and fissures. One or more compositions of the present invention can be prepared as additives to food, oral hygiene product, or any products having direct contact to an oral environment, especially an oral environment susceptible to dental caries or periodontal diseases. For instance, to treat or prevent dental caries or periodontal diseases compositions of the present invention can be formulated into a baby formula, mouthwash, lozenges, gel, varnish, toothpaste, toothpicks, tooth brushes, or other tooth cleansing devices, localized delivery devices such as sustained release polymers or microcapsules, oral irrigation solutions of any kind whether mechanically delivered or as oral rinses, pacifiers, and any food including, without limitation, chewing gums, candies, drinks, breads, cookies, and milk.

### EXAMPLES

The following examples are intended to illustrate but not to limit the invention in any manner, shape, or form, either explicitly or implicitly. While they are typical of those that might be used, other procedures, methodologies, or techniques known to those skilled in the art may alternatively be used.

In this study, we address this complex oral problem using Chinese medicinal herbs and herbal formulas. We used accurate oral microbiological assays to screen a large number of medicinal herbs that have exhibited clinical effectiveness. Some of the assays used in our studies are, first ever documented systematic screening herbal extracts. Through these studies, we discovered many useful bioactivities among the herbs screened. To our knowledge, we are the first group to combine various accurate oral microbiological assays to produce herbal formulas that have synergistic effects among chosen herbs and that provide a balanced approach to treat the complex bacterial infections of oral diseases. These new herbal formulas have great scientific and commercial values.

Oral pathogens do not remain as single cells, they form dental plaques which contain complicated bacterial flora in a biofilm. Successful treatments may need the ability to disrupt the dental plaque structure and to inhibit both Gram-positive and/or Gram-negative oral pathogens. At the same time, a successful treatment is also required to inhibit oral yeast infections since many anti-bacterial treatments make mucous membranes available for yeast infections. Furthermore, most non-harmful commensal bacteria in oral cavity should not be killed. Due to this complex situation, no single drug can effectively treat oral infection.

By understanding the fundamental biological mechanisms of oral diseases, we decided to develop herbal formulas that have the ability to provide balanced approaches to this complex problem. Our laboratory is one of the few laboratories that can accurately and effectively assay and analyze various aspects of oral microbial infections, including anti-bacterial/anti-fungal assays in liquid and solid culture, quorum sensing analysis in bacterial biofilm, species-specific recognition of oral pathogens using monoclonal antibody pathogen detection systems, direct imaging oral bacteria in saliva and dental plaque systems etc. We believe that we are the only laboratory to use these assays to screen over 400 Chinese Medicinal herbs.

Through these extensive analyses, we were able to find a list of herbs that exhibited various bioactivities. Furthermore, taking full advantage of synergetic efforts used in Traditional Chinese Medicine, we combined various accurate oral microbiological assays together to produce herbal formulas (F101 and F102) that provide a balanced approach to effectively treat the complex oral diseases.

Both formulas have strong killing effort on oral pathogenic bacteria or yeast, but less or no killing effect on other non-harmful, commensal bacteria. They are also capable of disrupting quorum sensing in bacterial biofilm. Tested with five different cellular assays, the formulas were found to have no cellular toxicity. The animal safety tests are on going. Based on our bench study and pre-clinical analyses, the herbal formulas can be used effectively against oral pathogens related dental diseases. The bioactivities in formula remain active in large-scale production and long time storage at room temperature. The testing for clinical effectiveness with animals and human subjects are on going.

### Example 1

### Selection and Preparation of Herbal Extract

Selection of Herbs

Chinese herbal medicine has well over three thousands years history. In excess of 5,000 Chinese herbs have been used to create and refine more than 100,000 formulas to fight various types of infections, illnesses and diseases. Through extensive statistical analyses between frequently used herbs (nearly 1500) and clinical effectiveness, we selected about 400 Chinese medicinal herbs for further analysis.

Preparation of Herbal Extracts

Each plant was extracted with both water-boiling and ethanol-soaking methods, in small and large scales.

For a common small-scale water boiling procedure, 50 g of an herb is mixed with 500 ml distilled water and boiling for up to 2 hours. The supernatant is precipitated with 60% ethanol at 4-degree overnight and then concentrated by evaporating ethanol and water. The stock solutions of herbal extracts are at 1 gram initial raw weight per 1 milliliter of water. The pH of the all extracts is adjusted to 7.0.

For a common small-scale ethanol-soaking procedure, 5 g of an herb is mixed with 50 ml 95% ethanol and incubated at room temperature for 3 days. The supernatant is then concentrated by evaporating ethanol under a vacuum. The stock solutions of herbal extracts are 1 gram initial raw weight per 1 milliliter of water. The pH of the all extracts is adjusted to 7.0.

A number of herbs with useful bioactivities have been prepared in large scale. For these preparations, similar experimental procedures are performed except in a larger volume in an industrial setting, usually 50 kg of an herb is mixed with 500 liters distilled water or 500 liters 95% ethanol.

For application in oral microbiological assays, each medicinal herb extract is diluted from stock solution, centrifuged at 3000 rpm for 10 min to remove left-over debris, and filtered through 0.2 micrometer filters to remove existing microbial particles.

### Example 2

### Herbal Extracts Against Gram Positive Cariogenic Bacteria

Mutans streptococci, lactobacilli and actinomyces are the known cariogenic bacteria. S. mutans, S. sobrinus, L. acidophilus, L. casei, L. plantarum, A. naeslundii, and A. viscosus are the most virulent cariogenic species among these bacteria. In this study, we screened the herbal extracts for the inhibitory effects against these gram positive bacteria using both liquid and solid plate culture assays.

For a standard liquid culture method, an herbal extract is sequentially diluted at a 1:2 ratio in 96 well plates with water (50 μl/well), then mixed with equal volumes of bacteria culture (1×10⁶ cells/ml) in Brain Heart Infusion (BHI) broth. After 24 hours incubation at 37° C., the growth of bacterial in each well is examined with a plate reader. The effect of the herbal extract on each tested bacterium is defined by the minimum inhibitory concentration (MIC) to prevent bacterial growth.

For a standard solid plate culture, an herbal extract is sequentially diluted at a 1:5 ratio and put into solid agar. A 5 mm diameter agar circle is then placed onto a bacterial lawn. The effect of the herbal extract on testing bacterium is defined by MIC to exhibit an inhibiting zone. Using these antibacterial assays, we found the following herbs that have anti-bacterial ability against these major cariogenic bacteria.

| | Inhibitory effect against cariogenic |
|---|---|
| Herb | bacteria |
| Rhus chinensis mill, | ++++ |
| Sophora flavescens Ait, | |
| glycyrrhiza uralensis Fisch | |
| Coptis chinensis franch, | ++ |
| perilla frutescens (Britt), | |
| Atractylodes chinensis koidz, | |
| Elsholtzia splendens | |
| Paris polyphylla Smith, | + |
| Prunus mume (sieb.), | |
| Amomum villosum, | |
| Sanguisorba officinalis, | |
| Eugenia caryophyllata, | |
| Bletilla striata (thunb), | |
| Amomum cardamomum (karvanh), | |
| Sophora tonkinensis (subprostrata), | |
| Melia toosendan | |

++++, MIC < 1.5 mg/ml; ++, MIC < 5 mg/ml; +, MIC < 50 mg/ml

### Example 3

### Herbal Extracts Against Gram Negative Periodontal Bacteria

Actinobacillus actinomycetemcomitants, Porphyromonas gingivalis, Fusobacterium nucleatum, Treponema denticola and Bacteroides forsythus are the most virulent Gram negative bacteria associated with periodontal disease. We screened the herbal extracts for the inhibitory efforts against these bacteria using the same methods described above. The result is listed below:

| | Inhibitory effect against periodontal |
|---|---|
| Herb | bacteria |
| Coptis chinensis franch | ++++ |
| Sophora flavescens Ait | |
| Rhus chinensis mill | ++ |
| glycyrrhiza uralensis Fisch | |
| perilla frutescens (Britt) | |
| Elsholtzia splendens | + |
| Atractylodes chinensis koidz | |
| Prunus mume (sieb.) | |
| Amomum villosum | |
| Sanguisorba officinalis | |

++++, MIC < 1.5 mg/ml; ++, MIC < 5 mg/ml; +, MIC < 50 mg/ml.

### Example 4

### Herbal Extracts Against Pathogenic Oral Yeasts

Candidas albicans, C. glabrata, C. guilliemondii, C. kefyr, C. krusei, C. stellatoidea and C. tropicalis are the most virulent yeast species related to yeast infection. C. albicans is the major oral yeast. We screened the herbal extracts for the inhibitory efforts against these pathogenic yeasts using the similar liquid and solid plate assays described above. The result is listed below:

| Herb | Inhibitory effect against oral yeast |
|---|---|
| Paris polyphylla Smith | ++++ |
| Sophora flavescens Ait | ++ |
| phellodendron amurense | + |

++++, MIC < 1.5 mg/ml; ++, MIC < 5 mg/ml; +, MIC < 50 mg/ml.

### Example 5

### Herbal Extracts Affecting Bacterial Quorum Sensing

Quorum sensing is a mechanism for bacteria to regulate gene expression in response to changes in population density. Many bacteria are capable of acyl-homoserine lactone based or peptides based intra-species quorum sensing and luxS-dependent inter-species quorum sensing. One feature regarding quorum sensing that has been extensively studied, is the link between quorum sensing and biofilm related gene expression.

There are several well-characterized examples for the involvement of intraspecies quorum sensing and biofilm formation. For example, lasI of Pseudomonas aeruginosa directs the synthesis of an acyl-homoserine lactone signal molecule used for P. aeruginosa intraspecies quorum signaling. Mutants in this gene were unable to produce biofilms that progressed beyond the very early stages of biofilm development. However, exogenous addition of the appropriate signal complemented the defect. A similar result was also obtained due to inactivation of the cep intraspecies quorum sensing system of Burkholderia cepacia.

Furthermore, a transposon mutagenesis study of the oral pathogen Streptococcus gordonii had detected a severe biofilm deficiency due to disruption of the two-component system required for its intraspecies quorum sensing system. In Staphylococcus aureus, intraspecies quorum signaling has been implicated as a negative regulator of biofilm formation.

In this study, we used an Agrobacterium tumefaciens based acyl-homoserine lactone quorum sensing reporter system and a Vibrio harveyi based luxS quorum sensing reporter system to screen herbal extracts. This is the first time that these systems have been used to screen herbal extracts.

A reporter gene system (traG::1acZ) of A. tumefaciens is used to perform acyl-homoserine lactone based quorum sensing response. One volume of overnight culture of the reporter strain is added to six volumes of sterile agar (0.7% in water, cooled to 45° C.). The suspension is mixed and layered over the surface of a petri dish (100 mm in diameter) containing 25 ml of culture agar medium with 40 micrograms of 5-bromo-3-indolyl-beta-D-galactopyranoside (X-Gal) per ml. Two microliters of the herbal formula is spotted onto the surface of the soft agar overlay. The results are observed after incubating the plate for 1 to 2 days at 28° C.

Induced expression of the reporter gene is measured semi-quantitatively. Positive and negative controls are included to ensure that the reporting system is working properly, and that the basal level expression of the reporter gene is below the detectable level. Development of blue color on the spotted area indicates a positive result, and the diameter of the color zone is used as a semi-quantitative measure of the observed activity. To exclude false-positive results that may be introduced from the herb extract, a control plate using heat-killed reporter strain is included in the experiment.

The effect of an herbal extract on LuxS mediated signal transduction is tested by examining the luminescence signal produced in the V. harveyi reporter strain BB170 or BB886. In the assay, 10 μl of herbal extract is sequentially diluted at 1:2 ratio in 96-well microtiter dishes. The V. harveyi reporter strain BB170 or BB886 is grown for 16 hr at 30° C. with aeration in AB medium and diluted 1:5,000 into fresh AB medium, and 90 μl of the diluted cells is added to the wells containing the diluted extract. Control wells contain 10 μl of distilled water. The microtiter dishes are inoculated at 30° C. Every hour, light production is measured by using a Wallac (Gaithersburg, Md.) Model 1450

| Herb | Effect |
|---|---|
| Sophora flavescens Ait, | Affect acyl-homoserine lactone based quorum |
| Medicinal rhubarb root | sensing |
| Coptis chinensis franch | Affect luxS based quorum sensing |

### Example 6

### Herbal Formulation F101 and F102

To produce an herbal formula that can provide multiple bioactivities for balanced treatment against oral infections, we mixed each herb listed under Example 2, with each herb listed under Example 3, with each herb listed under Example 4, and with each herb listed under Example 5. These resulting herbal formulas consist total of four herbs with one from each group under Example 2, 3, 4, or 5. These formulas are tested with bioassays listed in Examples 2, 3, 4, and 5.

Through the large scale screening of more than 1000 different combinations, we found a combination of Sophora flavescens Ait, Paris polyphylla Smith, perilla frutescens (Britt), and Coptis chinensis franch (F101) retained and even enhanced all bioactivities listed in Example 2, 3, 4, and 5. We also found that the combination of glycyrrhiza uralensis Fisch, Paris polyphylla Smith, perilla frutescens (Britt), and Coptis chinensis franch (F102) shows a majority of the desirable bioactivities except for the ability to affect acyl-homoserine lactone based quorum sensing.

To fine tune the bioactivities of F10, we varied the ratio of each herb extract from (1-5):(1-5):(1-5):(1-5) and found that 5:2:2:1 gives maximal effectiveness.

### Example 7

### Characterization of F101

To evaluate the consistency of the bioactivities in herbal extracts used in F101, we obtained herbs from four different locations (far north, east, south, and middle of China). The herbal extracts were prepared in large scale production (as described above) by four different Chinese herbal factories. The resulting herbal extracts were analyzed with HPLC and bioassays described in Examples 2, 3, 4, and 5. Our studies showed that herbs from different locations exhibited similar HPLC chemical profiles and similar bioactivities, demonstrating the consistency of F101 bioactivities.

To evaluate the stability of the bioactivities in herbal extracts used in F101, we have stored the F101 herbal extracts at different temperatures (4, 25, 37 and 60° C.) for over 18 months and still demonstrated over 90% bioactivities at all temperatures tested.

To evaluate the safety of F 01, the herbal formula was added to five different human cell lines including T cells, B cells, stem cells, epithelial cells and endothelial cells. No any negative effective was detected on cellular growth rate, cellular morphology, integrity of cell membrane, RNA or DNA. The F101 formula was also subjected to Ame's DNA mutagenesis tests, the results indicated that F101 did not induce any DNA point mutation, frame shift and other mutagenesis effects.

We tested the anti-microbial activities of F101 on not only the laboratory strains, but also the virulent clinical isolates from various human races (white, black, yellow etc). The results showed that F101 retained the potent effect against all virulent clinical isolates tested.

Using the patented monoclonal antibody based bacterial detection methods developed in our laboratory, we have the capacity to assay the killing effect of F101 on oral pathogens in real human saliva or saliva derived dental plaque. Our studies showed that F101 effectively kill all major oral pathogens existed in saliva and dental plaque, including Gram positive cariogenic bacteria such as S. mutans, S. sobrinus, L. acidophilus, L. casei, L. plantarum, A. naeslundii, A. viscosus, Gram negative periodontal bacteria such as Actinobacillus actinomycetemcomitants, Porphyromonas gingivalis, Fusobacterium nucleatum, Treponema denticola, Bacteroides forsythus and oral yeasts such as Candidas albicans. To the best of our knowledge, this is the first herbal formula that exhibits such broad inhibitory effects on all major oral pathogens in oral cavity.

More interestingly, about 50% commensal oral microorganisms in saliva and dental plaque survived the treatment of F101. These residual bacteria form a thinner dental plaque without acid-producing ability. This is ideal since these non-pathogenic bacteria may now occupy the ecologic niches thereby preventing new infections by pathogenic bacteria or yeasts. These data clearly demonstrate that F101 can provide a balanced treatment against oral infections and has huge potential applications in oral health care.

We believe that among other uses, F101 can be delivered as an additive to toothpaste, mouthwashes, chewing gum, or even baby formula. To ensure that the product development process will not affect the bioactivities of F101, we did a trial production of toothpaste in both gel and foam format with F101 added. The results showed that F101 in toothpaste exhibited the same bioactivities as regular solutions and that over 90% bioactivities were still detected after the toothpaste tubes were stored at room temperature for over 18 months. Additionally, fluoride compounds do not inhibit the antimicrobial activities of F101. We also tested F101 for staining and have clearly demonstrated that the mixture does not stain hydroxyapatite.

Although the invention has been described with reference to the presently preferred embodiment, it should be understood that various modifications can be made without departing from the spirit of the invention. Accordingly, the invention is limited only by the following claims.

What is claimed is:

1. A composition comprising a mixture of at least two components selected from the group consisting of phellodendron amurense, Paris polyphylla Smith, Prunus mume (sieb.), glycyrrhiza uralensis Fisch, Amomum villosum, Sanguisorba officinalis, Elsholtzia splendens, Eugenia caryophyllata, Rhus chinensis mill, Atractylodes chinensis koidz, perilla frutescens (Britt), Coptis chinensis franch, Sophora flavescens Ait, Bletilla striata (thunb), Amomum cardamomum (karvanh), Sophora tonkinensis (subprostrata), Melia toosendan, and Medicinal rhubarb root.

2. A composition comprising a mixture of at least three components selected from the group consisting of phellodendron amurense, Paris polyphylla Smith, Prunus mume (sieb.), glycyrrhiza uralensis Fisch, Amomum villosum, Sanguisorba officinalis, Elsholtzia splendens, Eugenia caryophyllata, Rhus chinensis mill, Atractylodes chinensis koidz, perilla frutescens (Britt), Coptis chinensis franch, Sophora flavescens Ait, Bletilla striata (thunb), Amomum cardamomum (karvanh), Sophora tonkinensis (subprostrata), Melia toosendan, and Medicinal rhubarb root.

3. A composition comprising a mixture of at least four components selected from the group consisting of phellodendron amurense, Paris polyphylla Smith, Prunus mume (sieb.), glycyrrhiza uralensis Fisch, Amomum villosum, Sanguisorba officinalis, Elsholtzia splendens, Eugenia caryophyllata, Rhus chinensis mill, Atractylodes chinensis koidz, perilla frutescens (Britt), Coptis chinensis franch, Sophora flavescens Ait, Bletilla striata (thunb), Amomum cardamomum (karvanh), Sophora tonkinensis (subprostrata), Melia toosendan, and Medicinal rhubarb root.

4. A composition comprising a mixture of
component A selected from the group consisting of Paris polyphylla Smith, Prunus mume (sieb.), glycyrrhiza uralensis Fisch, Amomum villosum, Sanguisorba officinalis, Elsholtzia splendens, Eugenia caryophyllata, Rhus chinensis mill, Atractylodes chinensis koidz, perilla frutescens (Britt), Coptis chinensis franch, Sophora flavescens Ait, Bletilla striata (thunb), Amomum cardamomum (karvanh), Sophora tonkinensis (subprostrata), and Melia toosendan, and
component B selected from the group consisting of Prunus mume (sieb.), glycyrrhiza uralensis Fisch, Amomum villosum, Sanguisorba officinalis, Elsholtzia splendens, Rhus chinensis mill, Atractylodes chinensis koidz, perilla frutescens (Britt), Coptis chinensis franch, and Sophora flavescens Ait.

5. A composition comprising a mixture of
component A selected from the group consisting of Paris polyphylla Smith, Prunus mume (sieb.), glycyrrhiza uralensis Fisch, Amomum villosum, Sanguisorba officinalis, Elsholtzia splendens, Eugenia caryophyllata, Rhus chinensis mill, Atractylodes chinensis koidz, perilla frutescens (Britt), Coptis chinensis franch, Sophora flavescens Ait, Bletilla striata (thunb), Amomum cardamomum (karvanh), Sophora tonkinensis (subprostrata), and Melia toosendan, and
component B selected from the group consisting of phellodendron amurense, Paris polyphylla Smith, and Sophora flavescens Ait.

6. A composition comprising a mixture of
component A selected from the group consisting of Paris polyphylla Smith, Prunus mume (sieb.), glycyrrhiza uralensis Fisch, Amomum villosum, Sanguisorba officinalis, Elsholtzia splendens, Eugenia caryophyllata, Rhus chinensis mill, Atractylodes chinensis koidz, perilla frutescens (Britt), Coptis chinensis franch, Sophora flavescens Ait, Bletilla striata (thunb), Amomum cardamomum (karvanh), Sophora tonkinensis (subprostrata), and Melia toosendan, and
component B selected from the group consisting of Coptis chinensis franch, Sophora flavescens Ait, and Medicinal rhubarb root.

7. A composition comprising a mixture of
component A selected from the group consisting of Prunus mume (sieb.), glycyrrhiza uralensis Fisch, Amomum villosum, Sanguisorba officinalis, Elsholtzia splendens, Rhus chinensis mill, Atractylodes chinensis koidz, perilla frutescens (Britt), Coptis chinensis franch, and Sophora flavescens Ait, and
component B selected from the group consisting of phellodendron amurense, Paris polyphylla Smith, and Sophora flavescens Ait.

8. A composition comprising a mixture of
component A selected from the group consisting of Prunus mume (sieb.), glycyrrhiza uralensis Fisch, Amomum villosum, Sanguisorba officinalis, Elsholtzia splendens, Rhus chinensis mill, Atractylodes chinensis koidz, perilla frutescens (Britt), Coptis chinensis franch, and Sophora flavescens Ait, and
component B selected from the group consisting of Coptis chinensis franch, Sophora flavescens Ait, and Medicinal rhubarb root.

9. A composition comprising a mixture of
component A selected from the group consisting of phellodendron amurense, Paris polyphylla Smith, and Sophora flavescens Ait, and
component B selected from the group consisting of Coptis chinensis franch, Sophora flavescens Ait, and Medicinal rhubarb root.

10. A composition comprising a mixture of
component A selected from the group consisting of Paris polyphylla Smith, Prunus mume (sieb.), glycyrrhiza uralensis Fisch, Amomum villosum, Sanguisorba officinalis, Elsholtzia splendens, Eugenia caryophyllata, Rhus chinensis mill, Atractylodes chinensis koidz, perilla frutescens (Britt), Coptis chinensis franch, Sophora flavescens Ait, Bletilla striata (thunb), Amomum cardamomum (karvanh), Sophora tonkinensis (subprostrata), and Melia toosendan,
component B selected from the group consisting of Prunus mume (sieb.), glycyrrhiza uralensis Fisch, Amomum villosum, Sanguisorba officinalis, Elsholtzia splendens, Rhus chinensis mill, Atractylodes chinensis koidz, perilla frutescens (Britt), Coptis chinensis franch, and Sophora flavescens Ait, and
component C selected from the group consisting of phellodendron amurense, Paris polyphylla Smith, and Sophora flavescens Ait.

11. A composition comprising a mixture of
component A selected from the group consisting of Prunus mume (sieb.), glycyrrhiza uralensis Fisch, Amomum villosum, Sanguisorba officinalis, Elsholtzia splendens, Rhus chinensis mill, Atractylodes chinensis koidz, perilla frutescens (Britt), Coptis chinensis franch, and Sophora flavescens Ait,
component B selected from the group consisting of phellodendron amurense, Paris polyphylla Smith, and Sophora flavescens Ait, and
component C selected from the group consisting of Coptis chinensis franch, Sophora flavescens Ait, and Medicinal rhubarb root.

12. The composition of claim 10 further comprising component D selected from the group consisting of Coptis chinensis franch, Sophora flavescens Ait, and Medicinal rhubarb root.

13. A composition comprising a mixture of Sophora flavescens Ait, Paris polyphylla Smith, perilla frutescens (Britt), and Coptis chinensis franch.

14. A composition comprising a mixture of glycyrrhiza uralensis Fisch, Paris polyphylla Smith, perilla frutescens (Britt), and Coptis chinensis franch.

15. A composition comprising a mixture of Elsholtzia splendens, Paris polyphylla Smith, perilla frutescens (Britt), and Coptis chinensis franch.

16. The composition of claim 13, wherein the weight ratio for Sophora flavescens Ait is from about 1 to 5, for Paris polyphylla Smith is from about 1 to 5, for perilla frutescens (Britt) is from about 1 to 5, and for Coptis chinensis franch is from about 1 to 5.

17. The composition of claim 13, wherein the weight ratio for Sophora flavescens Ait, Paris polyphylla Smith, perilla frutescens (Britt), and Coptis chinensis franch is about 5:2:2:1.

18. The composition of claim 14, wherein the weight ratio for glycyrrhiza uralensis Fisch is from about 1 to 5, for Paris polyphylla Smith is from about 1 to 5, for perilla frutescens (Britt) is from about 1 to 5, and for Coptis chinensis franch is from about 1 to 5.

19. The composition of claim 14, wherein the weight ratio for glycyrrhiza uralensis Fisch , Paris polyphylla Smith, perilla frutescens (Britt), and Coptis chinensis franch is about 5:2:2:1.

20. The composition of claim 15, wherein the weight ratio for Elsholtzia splendens is from about 1 to 5, for Paris polyphylla Smith is from about 1 to 5, for perilla frutescens (Britt) is from about 1 to 5, and for Coptis chinensis franch is from about 1 to 5.

21. The composition of claim 15, wherein the weight ratio for Elsholtzia splendens, Paris polyphylla Smith, perilla frutescens (Britt), and Coptis chinensis franch is about 5:2:2:1.

22. The composition of claim 13 further comprising a carrier.

23. The composition of claim 14 further comprising a carrier.

24. The composition of claim 15 further comprising a carrier.

25. A formulation suitable for topical administration comprising the composition of claim 1.

26. A formulation suitable for topical administration comprising the composition of claim 13.

27. A formulation suitable for topical administration comprising the composition of claim 14.

28. A formulation suitable for topical administration comprising the composition of claim 15.

29. An oral hygiene product comprising the composition of claim 1.

30. An oral hygiene product comprising the composition of claim 13.

31. An oral hygiene product comprising the composition of claim 14.

32. An oral hygiene product comprising the composition of claim 15.

33. A food additive composition comprising the composition of claim 1.

34. A food additive composition comprising the composition of claim 13.

35. A food additive composition comprising the composition of claim 14.

36. A food additive composition comprising the composition of claim 15.

37. A dried extraction elute of the mixture of claim 1.

38. A dried extraction elute of the mixture of claim 13.

39. A dried extraction elute of the mixture of claim 14.

40. A dried extraction elute of the mixture of claim 15.

41. The composition of claim 1, wherein the composition has an anti-microbial effect.

42. The composition of claim 2, wherein the composition has an anti-microbial effect.

43. The composition of claim 3, wherein the composition has an anti-microbial effect.

44. The composition of claim 1, wherein the composition has an anti-microbial effect on a cariogenic bacterium.

45. The composition of claim 1, wherein the composition has an anti-microbial effect on a microorganism selected from the group consisting of Gram positive bacteria, Gram negative bacteria, and yeast.

46. The composition of claim 41, wherein the composition has an effect on bacterial quorum sensing.

47. The composition of claim 1, wherein the composition has an anti-microbial effect on a microorganism selected from the group consisting of S. mutans, S. sobrinus, L. acidophilus, L. casei, L. plantarum, A. naeslundii, A. viscosus, Actinobacillus actinomycetemcomitants, Porphyromonas gingivalis, Fusobacterium nucleatum, Treponema denticola, Bacteroides forsythus, Candidas albicans, C. glabrata, C. guilliemondii, C. kefyr, C. krusei, C. stellatoidea and C. tropicalis.

48. The composition of claim 1, wherein the composition has an anti-microbial effect on S. mutans.

49. A method of inhibiting the activity of a microorganism comprising contacting the microorganism to a composition comprising a component selected from the group consisting of phellodendron amurense, Paris polyphylla Smith, Prunus mume (sieb.), glycyrrhiza uralensis Fisch, Amomum villosum, Sanguisorba officinalis, Elsholtzia splendens, Eugenia caryophyllata, Rhus chinensis mill, Atractylodes chinensis koidz, perilla frutescens (Britt), Coptis chinensis franch, Sophora flavescens Ait, Bletilla striata (thunb), Amomum cardamomum (karvanh), Sophora tonkinensis (subprostrata), Melia toosendan, and Medicinal rhubarb root.

50. The method of claim 49, wherein the composition comprises a mixture of at least two components selected from the group consisting of phellodendron amurense, Paris polyphylla Smith, Prunus mume (sieb.), glycyrrhiza uralensis Fisch, Amomum villosum, Sanguisorba officinalis, Elsholtzia splendens, Eugenia caryophyllata, Rhus chinensis mill, Atractylodes chinensis koidz, perilla frutescens (Britt), Coptis chinensis franch, Sophora flavescens Ait, Bletilla striata (thunb), Amomum cardamomum (karvanh), Sophora tonkinensis (subprostrata), Melia toosendan, and Medicinal rhubarb root.

51. The method of claim 49, wherein the composition comprises a mixture of Sophora flavescens Ait, Paris polyphylla Smith, perilla frutescens (Britt), and Coptis chinensis franch.

52. The method of claim 49, wherein the microorganism is an oral pathogenic microorganism.

53. The method of claim 49, wherein the microorganism causes dental caries or periodontal disease.

54. The method of claim 49, wherein the microorganism is on a mucosal surface.

55. The method of claim 49, wherein the microorganism is associated with a tooth structure.

56. The method of claim 49, wherein the microorganism is associated with an infection in an epithelial tissue.

57. A method of treating or preventing a microbial infection comprising administering to a subject in need of such treatment a composition comprising a component selected from the group consisting of phellodendron amurense, Paris polyphylla Smith, Prunus mume (sieb.), glycyrrhiza uralensis Fisch, Amomum villosum, Sanguisorba officinalis, Elsholtzia splendens, Eugenia caryophyllata, Rhus chinensis mill, Atractylodes chinensis koidz, perilla frutescens (Britt), Coptis chinensis franch, Sophora flavescens Ait, Bletilla striata (thunb), Amomum cardamomum (karvanh), Sophora tonkinensis (subprostrata), Melia toosendan, and Medicinal rhubarb root.

58. The method of claim 57, wherein the composition comprises a mixture of at least two components selected from the group consisting of phellodendron amurense, Paris polyphylla Smith, Prunus mume (sieb.), glycyrrhiza uralensis Fisch, Amomum villosum, Sanguisorba officinalis, Elsholtzia splendens, Eugenia caryophyllata, Rhus chinensis mill, Atractylodes chinensis koidz, perilla frutescens (Britt), Coptis chinensis franch, Sophora flavescens Ait, Bletilla striata (thunb), Amomum cardamomum (karvanh), Sophora tonkinensis (subprostrata), Melia toosendan, and Medicinal rhubarb root.

59. The method of claim 57, wherein the composition comprises a mixture of Sophora flavescens Ait, Paris polyphylla Smith, perilla frutescens (Britt), and Coptis chinensis franch.

60. The method of claim 57, wherein the microbial infection is an oral microbial infection.

61. The method of claim 57, wherein the microbial infection causes dental caries or periodontal disease.

62. The method of claim 57, wherein the microbial infection is on a mucosal surface.

63. The method of claim 57, wherein the microbial infection is associated with an epithelial tissue or tooth structure.

64. A method of preventing a microbial infection comprising contacting a composition to an area susceptible to a microorganism causing the microbial infection, wherein the composition comprises a component selected from the group consisting of phellodendron amurense, Paris polyphylla Smith, Prunus mume (sieb.), glycyrrhiza uralensis Fisch, Amomum villosum, Sanguisorba officinalis, Elsholtzia splendens, Eugenia caryophyllata, Rhus chinensis mill, Atractylodes chinensis koidz, perilla frutescens (Britt), Coptis chinensis franch, Sophora flavescens Ait, Bletilla striata (thunb), Amomum cardamomum (karvanh), Sophora tonkinensis (subprostrata), Melia toosendan, and Medicinal rhubarb root.

65. The method of claim 64, wherein the composition comprises a mixture of at least two components selected from the group consisting of phellodendron amurense, Paris polyphylla Smith, Prunus mume (sieb.), glycyrrhiza uralensis Fisch, Amomum villosum, Sanguisorba officinalis, Elsholtzia splendens, Eugenia caryophyllata, Rhus chinensis mill, Atractylodes chinensis koidz, perilla frutescens (Britt), Coptis chinensis franch, Sophora flavescens Ait, Bletilla striata (thunb), Amomum cardamomum (karvanh), Sophora tonkinensis (subprostrata), Melia toosendan, and Medicinal rhubarb root.

66. The method of claim 64, wherein the composition comprises a mixture of Sophora flavescens Ait, Paris polyphylla Smith, perilla frutescens (Britt), and Coptis chinensis franch.

67. The method of claim 64, wherein the microorganism is an oral pathogenic microorganism.

68. The method of claim 64, wherein the microorganism causes dental caries or periodontal disease.

69. The method of claim 64, wherein the microorganism is on a mucosal surface.

70. The method of claim 64, wherein the microorganism is associated with an infection in an epithelial tissue or tooth structure.

71. A method of treating or preventing a microbial infection comprising administering to a subject in need of such treatment a comprehensive anti-microbial composition, wherein the composition comprises an anti-G⁺ bacterial agent, an anti-G⁻ bacterial agent, an anti-fungus agent, and an agent capable of disrupting bacterial quorum sensing.

72. The method of claim 71, wherein the microbial infection is on a mucosal surface.

73. The method of claim 71, wherein the mucosal surface is selected from the group consisting of mouth, vagina, gastrointestinal tract, and esophageal tract.

74. The method of claim 71, wherein the microbial infection is an oral microbial infection.

75. The method of claim 71, wherein the microbial infection is associated with an epithelial tissue or tooth structure.

76. A method of inhibiting the activity of a microorganism comprising contacting the microorganism to a comprehensive anti-microbial composition, wherein the composition comprises an anti-G⁺ bacterial agent, an anti-G⁻ bacterial agent, an anti-fungus agent, and an agent capable of disrupting bacterial quorum sensing.

77. The method of claim 76, wherein the microorganism is an oral pathogenic microorganism.

78. The method of claim 76, wherein the microorganism causes dental caries or periodontal disease.

79. The method of claim 76, wherein the microorganism causes an infection associated with an epithelial tissue or tooth structure.

80. An herbal library consisting essentially of phellodendron amurense, Paris polyphylla Smith, Prunus mume (sieb.), glycyrrhiza uralensis Fisch, Amomum villosum, Sanguisorba officinalis, Elsholtzia splendens, Eugenia caryophyllata, Rhus chinensis mill, Atractylodes chinensis koidz, perilla frutescens (Britt), Coptis chinensis franch, Sophora flavescens Ait, Bletilla striata (thunb),

81. Amomum cardamomum (karvanh), Sophora tonkinensis (subprostrata), Melia toosendan, and Medicinal rhubarb root.

82. The herbal library of claim 80 further comprising an instruction.

83. A composition comprising a mixture of
component A selected from the group consisting of Paris polyphylla Smith, Prunus mume (sieb.), glycyrrhiza uralensis Fisch, Amomum villosum, Sanguisorba officinalis, Elsholtzia splendens, Eugenia caryophyllata, Rhus chinensis mill, Atractylodes chinensis koidz, perilla frutescens (Britt), Coptis chinensis franch, Sophora flavescens Ait, Bletilla striata (thunb), Amomum cardamomum (karvanh), Sophora tonkinensis (subprostrata), and Melia toosendan,
component B selected from the group consisting of Prunus mume (sieb.), glycyrrhiza uralensis Fisch, Amomum villosum, Sanguisorba officinalis, Elsholtzia splendens, Rhus chinensis mill, Atractylodes chinensis koidz, perilla frutescens (Britt), Coptis chinensis franch, and Sophora flavescens Ait, and
component C selected from the group consisting of Coptis chinensis franch, Sophora flavescens Ait, and Medicinal rhubarb root.

84. A composition comprising a mixture of
component A selected from the group consisting of Paris polyphylla Smith, Prunus mume (sieb.), glycyrrhiza uralensis Fisch, Amomum villosum, Sanguisorba officinalis, Elsholtzia splendens, Eugenia caryophyllata, Rhus chinensis mill, Atractylodes chinensis koidz, perilla frutescens (Britt), Coptis chinensis franch, Sophora flavescens Ait, Bletilla striata (thunb), Amomum cardamomum (karvanh), Sophora tonkinensis (subprostrata), and Melia toosendan,
component B selected from the group consisting of phellodendron amurense, Paris polyphylla Smith, and Sophora flavescens Ait, and
component C selected from the group consisting of Coptis chinensis franch, Sophora flavescens Ait, and Medicinal rhubarb root.
